(19) United States
(12) Patent Application Publication
(10) Pub. No.: US 2008/0114271 A1
(43) Pub. Date: May 15, 2008

(54) Method of neuromuscular calibration

(76) Inventor: David Rubenstein, Westminster, CA (US)

Correspondence Address: FULWIDER PATTON LLP, 6060 CENTER DRIVE, 10TH FLOOR, LOS ANGELES, CA, 90045, US

(21) Appl. No.: 11/598,399

(22) Filed: Nov. 13, 2006

(51) Int. Cl. A61B 5/00

(52) U.S. Cl. 600/595

(57) ABSTRACT

Assessing a patient to select an articulated body part to be calibrated and repeatedly moving the body part slowly back and forth through a selected path while yielding applying a low level restrictive force to movement in at least one direction along the path.

### BACKGROUND OF THE INVENTION

1. Field of the Invention

The invention relates to techniques for relieving pain and enhancing physical performance.

2. Description of the Prior Art

The intensity of pain dealt with by patients can range from minor irritations to totally debilitating symptoms.

Loss of muscular function often leads to pain as the patient loses muscle capacity causing symptoms of pain to often arise. The lost capability manifests itself in many ways, including loss of coordination, flexibility, strength and/or unreliability. The loss of capability is often referred to as neuromuscular inefficiency. The functioning of the neuromuscular system may be calibrated to restore or optimize the brain's function in controlling the patient's muscles. It is this restoration of structural integrity in the neuromuscular system to which my present invention is directed.

Attention has been given to the treatment of injuries by physical therapy and to strengthening body parts. It has been proposed to provide interactive devices for enhancing physical performance. It has been recognized that the human brain has the capacity to adapt and reorganize around an existing impediment to reestablish lost function. This mechanism is known as neuroplasticity. It has been proposed to improve body part performance by treating the patient with repetitive cycles of movement along specific paths through prescribed ranges of motion at predetermined speeds with the application of resistive or assistive forces. These procedures have been proposed for increasing the subjects capability in suppleness, in building strength and endurance. A device and method for developing ability in the neuromuscular system employing these concepts has been proposed in U.S. Pat. No. 7,066,896 to Kiselik. While likely providing some benefit in the maintaining of suppleness in the body tissue such as muscles and ligaments and in the building of strength, such a method has the shortcoming in that it does not recognize the segmented movements of a body part as symptomatic of a decalibration of the neuromuscular system.

Because of the discomforts and debilitation associated with pain, significant research and efforts have been made to deal with chronic pain. The field of pain management continues to grow at a rapid pace. Specialists have developed expertise in different fields of pain management. There are also varieties of pain treatment facilities which treat pain in different manners. Some focus solely on the psychological aspects of chronic pain and others treat pain with a behavioral approach. Other procedures focus on “functional restoration,” concentrating on exercise and physical therapy. Physical therapy is typically directed to the treatment of injuries and loss of muscle use due to surgery, accident or the like rather than being directed to the calibration of the neuromuscular system to remove the source of the flaw. As such, the therapist typically selects an established regimen. He or she will take the patient passively through selected motions of the articulated body part to detect the area and extent of the injury, typically not seeking to optimize the functioning of the neuromuscular system. Rather, a rigid procedure for treatment of the body part is established to secure functioning of the body part or strengthening thereof. This approach does little to address the pain associated with a flawed body part or to retrain the brain to lead to a reduction in pain or the like.

### SUMMARY OF THE INVENTION

My procedure recognizes that the kinetic chain incorporates all soft tissues involved in generating movement including muscle, tendon, ligament and fascia, as well as the nervous system and articular system. My procedure which I refer to as Neuromuscular Calibration, involves the programming of the kinetic chain into neuromuscular efficiency through a protocol of micro motion exercises using a resistance load applied during the eccentric and concentric phase in the motion.

My procedure is believed to restore the optimal synaptic firing patterns in the brain to improve neuromuscular efficiency, physical and athletic performance. It is believed that over time, the neuromuscular system becomes decalibrated leading to the brain operating below its normal standard of synaptic discharge coherency. The observation of aberrant, inefficient motion within the kinetic chain, reveals the absence of calibration in the brain. The brain is believed to possess a perfect motion template reflecting the brain's precise awareness of the relative strength, flexibility, coordination and balance of the body parts. This perfect motion template contains the code for the greatest neuromuscular efficiency of synaptic firing. Over time, a variety of factors such as injuries and disuse tend to create a dissonance between the perfect motion template and the reality of how the body actually performs. In decalibration, a blurring effect occurs or is correlated to a loss of focus by the brain on the perfect motion template leading to such decalibration.

I have discovered that the kinetic chain may be utilized as a tool in Neuromuscular Calibration to evaluate the level of performance of the brain and the processing of motion information, i.e. the management of motion. Rather than dealing directly with the kinetic chain at the location where the symptoms manifest themselves, my procedure involves accessing brain function directly thereby opening a domain of relatively untapped potential. A reliable indicator of decalibration is broken motion where the motion lacks fluidity, interrupted by small starts and stops, pauses or hops which may be described as a ratcheted motion creating a segmented or stuttered quality to the motion, opposite to graceful motion. Decalibration can result from the fact that the body parts were never developed well in the first place or from accident, misuse, overuse, abuse, or disease, extreme fatigue, dehydration, poisons or toxins, drugs and venoms or delayed onset of muscular soreness. I have discovered that in moving a flawed body part, such as a part of a foot, knee, hip, neck or shoulder through a normal path of articulation, such as in an arc, against a relatively modest and yielding resistive force, as applied by a technician's hands, a somewhat segmented or ratcheted motion can often be detected thus indicating a decalibration of the neuromuscular system serving that body part. The technician or “calibrationist” may resist the movement with a relatively small force and in so doing can visually observe and physically feel a stutter or ratcheting in the patient's movement of that body part thus indicating limitations or malfunctions existing in the motor and sensory cortex of the brain and/or the cerebellum.

The information relating to the extent and degree of decalibration can be detected and recorded to assist in performing an effective procedure and for future reference. It is believed that the loss in ability for regular and smooth movement of an articulated body part may produce many forms of pain resulting in impaired coordination, flexibility, weakness, or unreliability. This neuromuscular inefficiency is termed “decalibration.” The purpose of my technique is to optimize the brain's control over the muscles involved to achieve restoration of structural integrity and elimination of the symptoms such as pain associated with decalibration.

Assessment can reveal a reduction in command and control of the finer motions. The technician may assess the extent of decalibration by observing the patient's posture and balance of movement during a walking exercise. My method involves a two-part process of assessment and active (often palmer) micro exercises and mobilization techniques. These micro exercises both reveal (1) stuttered or segmented movement and (2) optimize neuromuscular efficiency may be observed and/or felt by the technician and usually occurs during the eccentric phases (elongation of the muscles) of motion.

In some embodiments of my invention, I apply numerical indicators to the detected degree of decalibration (segmented motion) so that each segmented motion may be assigned a numerical value such as a “−3” for an exaggerated segmented motion, “−2” for a large segmented motion, a “−1” for a medium segmented motion and a “−0.5” for a small magnitude segmented motion and a “00” for a smooth, strong, full range of movement under convincing power. It is the object of my process to optimize the forces produced by the neuromuscular system and to optimize the body's capability to receive and deal with forces encountered in every day living and athletic activities.

For various applications and treating the afflictions of various different patient's, I select multiple exercises of the above steps to address the particular issue involved. In any event, at the conclusion of my treatment, I typically seek to program the results into the patient by removing the patient to a somewhat spacious area, whether indoors or outdoors, and direct that patient to walk a number of steps, for instance, 10 to 15 steps, away from me and then back toward me in a straight line so that I can observe a patient's movement and possibly detect any correction in what was previously asymmetrical movements. I might record those improvements and will further instruct the patient to repeat the walking steps two or three more times, a technique which I find is effective to program the patient and minimize recidivism.

Proprioception is the organic control mechanism for coordinating motion of the body parts, equilibrium and posture, whether it be micro posture such as the degree of precision in which two bones meet or macro posture as when the body is viewed as a whole as seen in a standing or sitting position. It is recognized that too much or too little tension will create musculoskeletal problems, poor balance and weak physical performance. I have found that an analysis of the patient's posture and irregularity during walking is a prime tool for evaluating any dysfunction. Walking can be very revealing with respect to detail and clarity for most musculoskeletal issues, when the proper analysis is utilized to collect the relevant details. For example, the impact of the heel, rolling motion of the foot through the stride and how much spring (energy return) can be observed in the arches. Thus, by analyzing these details the technician or calibrationist can detect telltale signs indicating flaws in certain areas which suggest some degree of decalibration. The procedure of the present invention focuses on the capability of the brain to control the musculoskeletal motion and seeks to re-teach the brain to overcome defections in a manner which has a significant degree of permanency.

In the brain, neurons connect to other neurons by way of branches. These connection points, combined with the electrical firing sequence, constitutes the matrix by which propriception is generated in the brain. Calibration to restore the optimal synaptic firing patterns in the brain to produce peak neuromuscular efficiency, physical and athletic performance tends to minimize the ratcheting and segmented movement. Decalibration suggests that the brain is operating below its normal standard of potential of synaptic discharge coherency. The observation of aberrant, inefficient motion within the kinetic chain is indicative of the absence of calibration in the brain.

In my procedure, I seek to tap brain power. It is recognized that the neuro-net of the human brain is a bio-electric chemical-magnetic wonder of immense complexity. The brain produces some 50,000 individual chemicals, consciously computing roughly 2,000 calculations per second and unconsciously computing some 400 billion calculations per second. This organ is available to reorganize motions at a higher level in response to the procedure of the present invention.

The procedure of the present invention focuses on the brain and its biology. It recognizes that the kinetic chain is not the force or cause of most biomechanical inefficiencies. It recognizes that the efficiency of motion and movement is hard-wired into the brain. Efficient motion corresponds to coordination, grace, power and painless function. The brain already possess the patterns for perfect and efficient movement. These patterns are stored as permanent, pre-coded, instructions that can be readily accessed. My process accesses this memory via motions of linear, radial, concentric and/or eccentric nature and utilizes the active and passive muscular contraction to essentially reprogram. It is believed that once accessed, these synaptic firing patterns “re-set” or calibrate in such a manner as to restore optimal neuromuscular efficiency of the entire kinetic chain in question.

There is a fundamental difference between exercise and the Neuromuscular Calibration process. Exercise focuses on building and maintaining muscle by working the musculoskeletal system. The purpose of my process, Neuromuscular Calibration, is to work the brain and form new firing patterns which in turn enhance proprioception (balance and stability). For example, by training the brain to activate flacid musculature and to deactivate muscles in spasm, balance may be immediately achieved with respect to the physical forces moving through the body.

Various embodiments of the procedure of the present invention serve to restore greater range of motion around a body joint, optimize muscle engagement (tone), to minimize or eliminate pain, all in a relatively permanent manner. The procedure is different from physical training since it is intended to quickly calibrate the subject's neuromuscular circuit thus producing enhanced power, agility and flexibility.

My procedure relies on relatively minor movements which I refer to as micro exercises to engage the brain intensely. These exercises involve the movement of a body part over just a short distance or short arc and involves a low force of resistance supplied by the technician, typically on the order of 10% of the maximum force the patient could generate in the particular movement or micro exercise. Application of this low force of resistance to be resisted by the patient as the body part is moved back and forth along a selected path tends to generate a greater amount of neurological information than would be the case if higher forces were applied. In fact, higher forces tend to mask decalibration and for this and safety reasons are avoided as a means for my process.

During a given micro exercise, it is common to find muscles which are in spasm or inactive. The Push-Back Mobilization Technique works instantly for both of these muscle states. This technique causes a spasm to return to normal tone levels relieving pain and for an inactive muscle, it reactivates to its normal tone and responsiveness to the brain.

To better understand the Push-Back Mobilization Technique, I will use a foot calibration micro exercise to illustrate. During a foot calibration one of the micro-exercises is “toe curls.” The toes are held by the Calibrationist fingers providing an object for the toes to push against. A pushback is performed with the toes in full dorsi-flexion or all the way back. The client/patient begins a light force pushing the toes forward. Just after the force begins, the Calibrationist rapidly ‘pushes back’ the toes to their maximum ROM. This results in increased power and ROM instantly.

It is well understood that the eccentric phase of any muscular contraction produces approximately three times the force of the concentric phase. This would suggest that more neurological and brain activity and greater force production and greater collateral muscular contractions are involved than is the case with concentric contractions.

As a preliminary step, I seek to ascertain the nature and extent of musculoskeletal dysfunction by referring to the patient's medical history, examination and through testing and observation. In this manner, biomechanical dysfunctions may be identified and recorded in the patient's history. Thereafter, the process will proceed with passive movement of the subject's articulated body parts to prime the brain in stimulating the specific neuro pathways which would best serve to achieve the desired and proper motion as in the end result. Warm up is then followed by light active motion, passive stretching and palmer compressions by the technician. These steps serve to unlock imbricated osseous tissues to provide for increased spacing in the joint. During the calibration step, I typically take advantage of both concentric and eccentric motions under palmer loading of about 10% of the maximum force which could be overcome by the patient for that particular motion in the concentric movement. I typically focus on the eccentric phase as it is believed to be when the most brain neurons fire and has proven to produce the most telling indicators of decalibration, i.c. segmented motion indicating compromised biomechanics. Calibration may be enhanced by progressively higher exercise intensity with concentric/eccentric work in order to induce the strength of calibration. Once the calibration is completed, testing may demonstrate that previously impossible or flawed or otherwise compromised neuromuscular structure has been corrected and normal or optimal motion has been achieved. It is helpful to program the calibration by instructing the patient to walk 10-15 or so steps to set the calibration for a greater degree permanency.

From the foregoing, it will be appreciated that the method of the present invention may be characterized by the evaluation of a patient's articulated body part to be treated, such as the patient's foot, by subjecting it to passive warm up typically manipulating the body part through a few repetitions of movement to prime the brain and stimulate the specific neuro paths which are to be burdened with the calibration. The movement of the body part may be initially tested to identify any segmentation or ratcheting to the movement and again tested at the end of the procedure for a comparison to determine the extent to which such segmented movement has been eliminated to thus provide an objective indication of the degrees of success achieved.

Other features and advantages of the invention will become apparent from the following detailed description, taken in conjunction with the accompanying drawings which illustrate, by way of example, the features of the invention.

### BRIEF DESCRIPTION OF THE DRAWINGS

FIG. 1 is a diagram of steps which may be performed in one embodiment of the method of the present invention;

FIG. 2 is a bottom view of a patient's foot exhibiting target areas to be examined in the initial evaluation;

FIG. 3 is a side view of a patient's foot, leg supported by a stool at the calf, to be moved in an arc under a low level resistance force on the ball of the foot, pushing the ball of the foot concentrically away from the knee from a starting position of dorsi flexion to full plantar flexion, then from full plantar flexion eccentrically to dorsi flexion;

FIG. 4 is a side view of a patient's foot, leg supported by a stool at the calf, to be moved in an arc under a low level resistance force on the top of the foot, pulling the foot concentrically toward the knee from a starting position of full plantar flexion to dorsi flexion. From dorsi flexion, overcome the patient's force and move foot eccentrically to full plantar flexion;

FIG. 5 is a top view of a patient's foot, leg supported by a stool at the calf, to be moved in an arc under influence of a low level resistance force with the starting position of the foot in invert position and moving to evert concentrically, then from the evert position, eccentrically to the invert position;

FIG. 6 is a dorsal view of a patient's foot, leg supported by a stool at the calf, to be moved in an arc under influence of a low level resistance force acting laterally as the foot is moved from the evert position to invert concentrically then from the invert position, eccentrically to the evert position;

FIG. 7 is a dorsal view of a patient's foot, leg supported by a stool at the calf, to be moved in an arc under a low level resistance force acting in the plantar direction with the starting position of the foot in evert and moving to invert concentrically, then eccentrically to the evert position;

FIG. 8 is a perspective top view of a patient's foot, leg supported by a stool at the calf, the forefoot held stationary, the heel or aft foot moving independently from a start position of invert moving to a finish position of evert. From the evert position, overcome the patient's force and move foot eccentrically to the invert position;

FIG. 9 is a perspective top view of a patient's foot, leg supported by a stool at the calf, with the plantar surface on the ball of the foot pushed against the technician's knee in order to facilitate rounding of the plantar surface of the foot, start position is toes curled over the technician's knee, motion is concentric toward the patient's knee, eccentric away from the patient's knee;

FIG. 10 is a perspective oblique view of a patient's foot, leg supported by a stool at the calf, the plantar surface pushed against the technician's tibia and knee in order to facilitate stability, start position is toes dorsi flexed, motion is to curl toes concentrically toward the technician's knee, eccentric returning toes to start position with motion away from the technician's knee;

FIG. 11 is a perspective oblique view of a patient's foot, leg supported by a stool at the calf, also supported on the plantar surface with an emphasis on the foot pushed against the technician's tibia and knee in order to facilitate stability, and fingers holding toes rigid and straight, start position is toes dorsi flexed, motion is concentric toward the technician's knee, eccentric is away from the technician's knee. Keep toes straight for duration of exercise;

FIG. 12 is a side view of a patient's foot, leg supported by a stool at the calf, to be moved in an arc augmented by a low level, yielding resistance force with the starting position of the foot in invert position and moving to evert concentrically, then eccentrically to the invert position;

FIG. 13 is a plantar view of a patient's foot, leg supported just above the ankle by the opposite leg, foot to be moved in an arc at the ankle under a low level resistance force with the starting position of the foot in invert and moving to evert concentrically, then eccentrically to the invert position;

FIG. 14 is a plantar view of a patient's foot, leg supported just above the ankle by the opposite leg, foot to be moved in an arc at the ankle augmented by a low level resistance force with the starting position of the foot in evert and moving to invert concentrically then eccentrically to the evert position;

FIG. 15 is a lateral view of the patient's head, supported by the technician's opened hands, with fingers placed along the patient's jaw bilaterally, patient moves head first concentrically to the right under yielding resistance by the technician and then the technician overcomes the patient's force and turns the head under patient's yielding force eccentrically to the left. Process is then reversed so that patient moves head first concentrically to the left under yielding resistance by the technician and then the technician overcomes the patient's force and turns the head under patient yielding force eccentrically to the right for balance;

FIG. 16 is a superior view (from the top) of a seated patient with the arm suspended, palm up, elbow slightly bent, starting position is arm to the side and slightly behind the shoulder and parallel to the floor, patient moves the arm forward (anterior and concentric) under a yielding resistance by the technician until the arm crosses over the patient's centerline or sternum. Then the technician slightly overcomes the patient's low force and slowly pulls the patient's arm at the wrist rearward in an arc for the posterior and eccentric phase of movement;

FIG. 17 is a perspective view showing a standard hand to hand grip as in a patient and technician shaking hands, the patient starting in full pronation of the hand (palm faces downward) and rotating the hand toward supination (concentric) while the technician provides light resistance, once the patient has reached the end range of motion without discomfort, the technician initiating a reversal of the motion to supinate the patient's hands under a low force yielding resistance to first provide concentric supination and then concentric pronation of the patient's hand;

FIG. 18 shows a patient is seated with arms outstretched and draped over a wooden rod (broomstick works well) with their body turned to the right for the starting position (concentric motion is toward the left), patient rotates trunk under light yielding resistance from the technician until the end range of motion to the left has been reached, followed by the technician pushing in the reverse direction (eccentric) slightly overcoming the patient's force, rotating right and toward the starting position. Reverse process to calibrate other side;

FIG. 19 shows a patient is prone with legs together, leg is moved laterally under low level resistance force from the medial starting position (concentric). Then from the lateral position, overcome the patient's force and move the leg eccentrically to the medial position; and

FIG. 20 shows a patient in a seated position performing extension at the knee (concentric) against a low resistance yielding force by the technician until full lock is reached at the knee, then the technician applies a slightly increased force to reverse the arc (eccentric) pushing the patient's knee into flexion under a light yielding resistance.

### DETAILED DESCRIPTION OF THE PREFERRED EMBODIMENTS

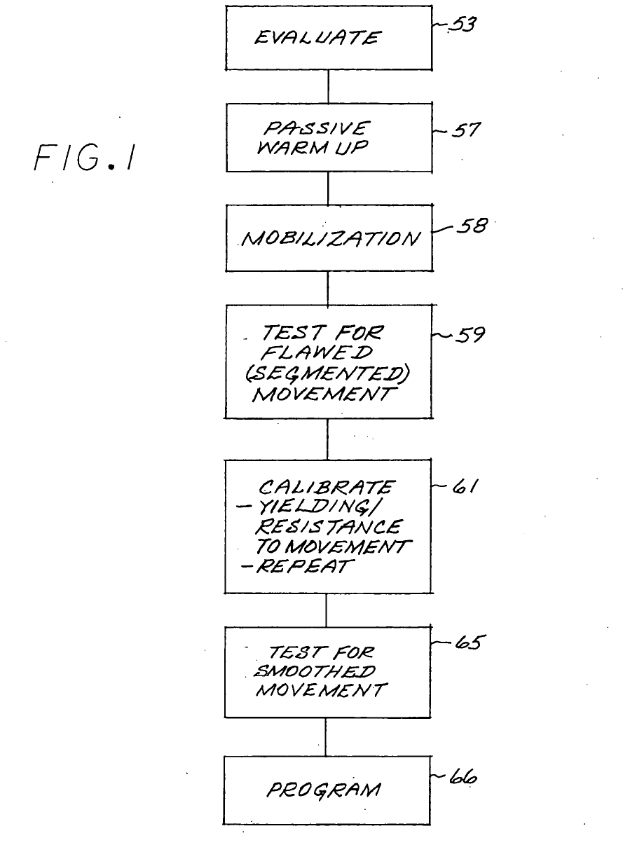

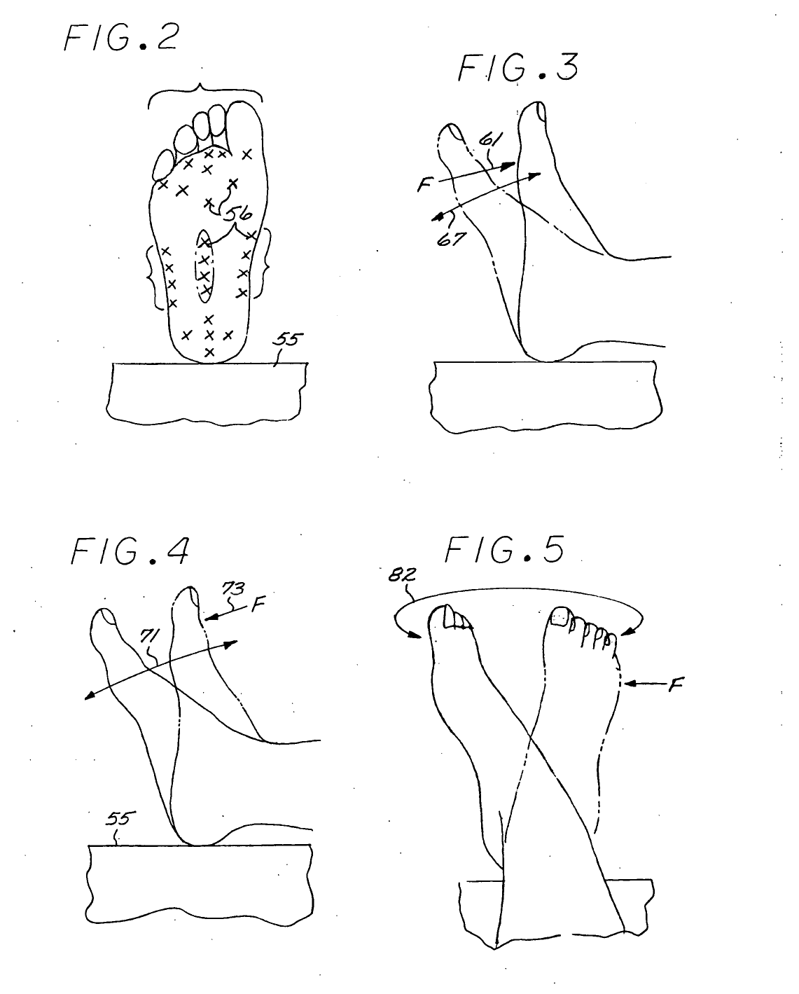

The procedure of the present invention may be applied to various body parts such as hands, feet, knees, hips, neck, and torso. In treating, for instance, a patient's foot 51 the technician will typically first evaluate the patient as in step 53 (FIG. 1), take the medical history and seek to determine the best approach for achieving Neuromuscular Calibration. I will describe the treatment of one of the patient's feet, it being understood that the other foot may then be treated in a similar manner. The evaluation may detect one foot coming down harder than the other in normal walking to indicate a neuromuscular inefficiency in that foot. The patient's feet will be examined physically to observe contour, shape, height, length and definition of the arches. For some patient's, my examination and evaluation of the foot 51 includes the palpation of the plantar surface at selected locations across and lengthwise of the foot as at the locations 56 shown in FIG. 2. Palpation is usually done with by the technician's thumb using a moderate pressure. Inquiry is made to the patient at each point as to the degree of pain or discomfort experienced from pressing at each point to assist in providing an indication of the nature and extent of musculoskeletal dysfunction(s).

The foot may be subjected to a passive warm up 57 (FIG. 1), by placing the foot on a support stand 55 (FIG. 3) and massaging and manipulating various foot parts with respect to others by hand manipulation to prime the brain by supporting and structuring the specific neuro pathways. As an optional step, the foot may then be mobilized as at 58 (FIG. 1) by instructing the patient to make light movements, applying pressure, stretching and palmer compression to stimulate the osseous tissue and increase the joint space. To obtain a calibration index for the plantar flexion, the technician may then subject the foot to a test at 59 (FIG. 1) by applying a low level force as depicted by the arrow 61 to the plantar surface, first on the lateral and then the medial side, asking the patient to move the forefoot forward and aft in the plantar direction against, or augmented by the technician's yielding force. The technician can typically feel and sometimes even observe irregularities in the movement of the foot which manifests itself as sort of a ratcheting or segmentation of the movement. The greater the segmentation, the more serious the decalibration is of the neuromuscular system. This procedure may be repeated for different parts and different movements of the foot to obtain somewhat of a baseline of the degree of decalibration and that information recorded in the patient's records.

Then, the technician may start with calibration of the foot as at 61 (FIG. 1) requesting the patient to move the forefoot forward and aft in the plantar and dorsi directions from the vertical solid line position to a distal broken line position as viewed in FIG. 3, all the while maintaining a yielding force on the plantar surface perpendicular to the radius from the axis of rotation in the plantar direction and directing the patient to resist the force both during the movement in the plantar and dorsi directions. This step is repeated slowly a number of times, on the order of 10, 15 or possibly up to 20 times at a repetition rate on the order of 15-20 and preferably about 15 repetitions in a 20-30 second period to achieve the calibration effect. As the technique is performed, the technician will actually feel or see the motion of the foot smooth out thus giving an indication that the neuromuscular system is becoming calibrated and that the brain has been somewhat retrained in controlling the motion of the foot. The technician may finally then test at 65 (FIG. 1) by maintaining the yielding force upwardly on the plantar surface in the forefoot area to resist distal movement of the forefoot and observe any ratcheting or segmentation in that motion, particularly as the forefoot flexes slowly proximal. The degree to which the segmented movement had been reduced or even eliminated is an indicator of the success achieved by the calibration process. After I have completed the number of exercises selected for the affliction of the particular patient, to help set the calibration for greater permanency, I may ask the patient to walk in a natural manner on the order of 10-15 or 20 steps to program the calibration as at 66 (FIG. 1) to provide greater resistance to decalibration. This programming will typically take place after treatment of several of the steps as described below.

When the neuromuscular system becomes decalibrated or less efficient, the affected body part(s) quickly lose their coordination, strength and grace oftentimes resulting in pain to the patient when the body part is moved or even sometimes at rest. The extent of decalibration can often be detected by the patient moving the body part through a particular motion such as an arc against a yielding force, such as the force applied by the hand of the technician to observe any segmentation in the motion, particularly during the eccentric phase of the motion when the muscles are lengthening. In this regard, the technique involves the application of relatively light loads, as for instance 10% of the maximum load that might otherwise be resisted or carried by the patient's movement of the particular body part. That is, in the movement of the foot 51 in the plantar direction, the patient may be capable of moving a maximum weight of 25 lbs. While my procedure will be effective with different magnitudes of force, it is most effective when the operative force applied by the technician is about 10% of the maximum that could otherwise be carried by the body part, i.e. about 2.5 lbs. It will be appreciated by applying the force manually, the technician can vary the amount of force applied so as to not overcome the strength of the patient but to keep it in the range of about 5-15% of the maximum force the patient could overcome in the various locations along the path of travel.

As an example, in a bicep curl, the technician may hold the patient's elbow to stabilize the arm and hold the patient's hand and resist the curl with a force approximately 10% of the maximum that could be lifted by the patient. The same technique during uncurling while the muscles are extending (eccentric) while resisting the load and may be more effective in detecting decalibration. Typically, the technician will actually feel segmentation in the motion as the patient curls or uncurls his or her arm thus allowing the technician to perceive the magnitude of segmentation indicative of the degree of decalibration. A smooth uncurling suggests the involved muscle has been calibrated.

In assessing the patient, I have found that the patient's movements in making walking steps may be highly indicative of the degree of decalibration afflicting the patient. A particularly effective approach for detecting any lack of symmetry in the walking motion is to instruct the patient to first walk 10-15 steps directly away from the calibrationist and then the same distance back toward the calibrationist. An extremely large decalibration usually corresponds to a history of fracture or significant ballistic impact which has resulted in an injury to the affected body part. A large but lesser magnitude of ratcheted or segmented motion is usually indicative of a lesser original injury and lesser severities. The lesser the magnitude of ratcheted motion, as is most common for many patients, indicates a medium decalibration while a small magnitude of ratcheted motion indicates an even lesser degree of decalibration.

To continue calibrating, the neuromuscular system controlling movement of the forefoot optionally move on to a dorsi flexion as shown in FIG. 4. Here I instructed the patient to move the forefoot fore and aft forth in the plantar and dorsi directions through the arc defined by the directional arrow 71 from the solid line position shown in FIG. 4, to the broken line position while applying a yielding resistive force depicted by arrow 73 to the dorsal surface of the forefoot. Again, as the forefoot is moved fore and aft through its arc, the technician applies a yielding force as depicted by the force arrow 73 of approximately 2.5 lbs., to the dorsal surface. The foot movement might then exhibit a ratcheted or segmented motion thus indicating decalibration. Again, the magnitude of decalibration may be felt or visually observed and an index indicative of the magnitude recorded in a patient's records.

With continued reference to FIG. 4, I then proceed with the calibration of the neuromuscular system controlling the foot in the dorsal direction step 61. The magnitude of force 71 is maintained on the order of about 10% of the total force a patient could overcome in moving his or her forefoot in the dorsal direction as indicated. Assuming the patient could lift 15 lbs. maximum, I apply about 1.5 lbs. to the dorsal surface thus yieldingly resisting the patient's voluntary movement of the forefoot as the foot is moved in one direction and augmenting movement of the opposite direction. I continue this approach and direct the patient to repeat the motion for 5 to 10 repetitions in some instances may be 15 up to 20 repetitions which may be necessary to provide a fairly complete calibration. After completing the selected number of repetitions, the technician will make a point of directing the patient to once more cycle the foot through the defined arc while maintaining the yielding load thereon and while the forefoot travels proximally observing any segmentation and the extent thereof so as to record data indicative of the extent of segmentation at the completion of the calibration, step 65 (FIG. 1).

It will be appreciated from the above and the foregoing, the patient's foot may be first subjected to plantar flexion, dorsi flexion, foot sweep, 90° dorsi lateral flexion, starting from the foot invert position and then the evert position and then steps involving 0° plantar lateral flexion, again first from the invert position and then from the evert, and then move through steps of fixing the heel while rotating the forefoot medially and laterally, starting first with the invert and then the evert positions, then fixing the forefoot and rotating the heel about the forefoot, first from the invert and then the evert positions. It will be appreciated by those skilled in the art that there is nothing particularly critical about the sequence in which these procedures are performed and that selected ones of the procedures will be required for some applications and others for different applications. In any event, the technician may then proceed with a procedure involving placement of the heel or ball of the foot against a solid surface and curling and uncurling the toes in the plantar direction against a resistive force, followed by the folding the toes in, and unfolding from the plantar direction against a resistive force, then extending the toes in the dorsal direction and flexing the foot.

As will be appreciated by those skilled in the art, the method for treating each of the articulated body parts typically involves concentric and eccentric motions under palmer loading to yielding load the body part in the direction of the tangent to the direction of the movement. I typically focus more heavily on the eccentric phase of the motion. As it is believed that more brain neurons fire during that phase.

I have found it helpful to, in some instances, perform one or more of the steps listed in the following Chart of Steps:

CHART OF STEPS
Exercise

| | |
|---|---|
| A. | Foot Sweep |
| B. | Reverse Foot Sweep |
| C. | Plantar Flexion |
| D. | Dorsi Flexion |
| E. | 90° Dorsi Flexion and Invert to Evert |
| F. | 90° Dorsi Flexion and Evert to Invert |
| G. | 0° Plantar Lateral Flexion Evert to Invert |
| H. | 0° Plantar Flexion Invert to Evert |
| I. | Forefoot Isolated Invert to Evert |
| J. | Forefoot Evert |
| K. | Aft Foot Invert |
| L. | Aft Foot Evert |
| M. | Toe Curls |
| N. | Toe Folds |
| O. | Dorsal Toe Extension |
| P. | Big Toe Knuckle Press |
| Q. | Crescent Plantar |
| R. | Crescent Dorsi |
| S. | Crescent Fold 90°, 0° |
| T. | Crescent Fold, 0° |
| U. | Ankle Rotations Invert to Evert |
| V. | Ankle Rotations Evert to Invert |
| W. | #1 Toe Abductions |
| X. | #5 Toe Abductions |
| Y. | Toe Adductions |
| Z. | Posterior Tibialis, Whole Leg |
| AA. | #1 Toe Abduction |

As described below the technician may subject the big toe to plantar flexion, press the knuckles of the various toes and then continue instructing the patient to bend his or her foot into sort of a crescent shape, then while maintaining the crescent shape, proceed with plantar flexion of the foot, and then in the dorsal direction while holding the foot in the crescent posture. The procedure may also then include the ankle rotations, toe abduction and the like and finally conclude with a test of the foot movements in each phase to detect the magnitude of any segmented movement for comparison with the initial assessment to determine the extent of progress. It will be appreciated that each of the foregoing articulations will be resisted by a low level yieldingly resistive force against the patient's contraction or extension of his or her muscles controlling movement of the articulated body part.

Referring to FIG. 5, I may then move to the step of calibrating the 90° plantar lateral flexion invert with the patient's foot overhanging the support 55 and the foot initially in the invert position with the plantar surface facing medially in the direction of the directional arrow 82. I apply the usual yieldingly resistive forces to that movement so I can detect any segmentation suggesting calibration and record the index associated therewith.

This sweeping motion from the invert to the evert position will be continued again for a number of repetitions, in the order of 5 to 10 or maybe up to 20. At the conclusion, the index associated with the degree of segmentation will be recorded to thus maintain a record of the degree of success in calibration.

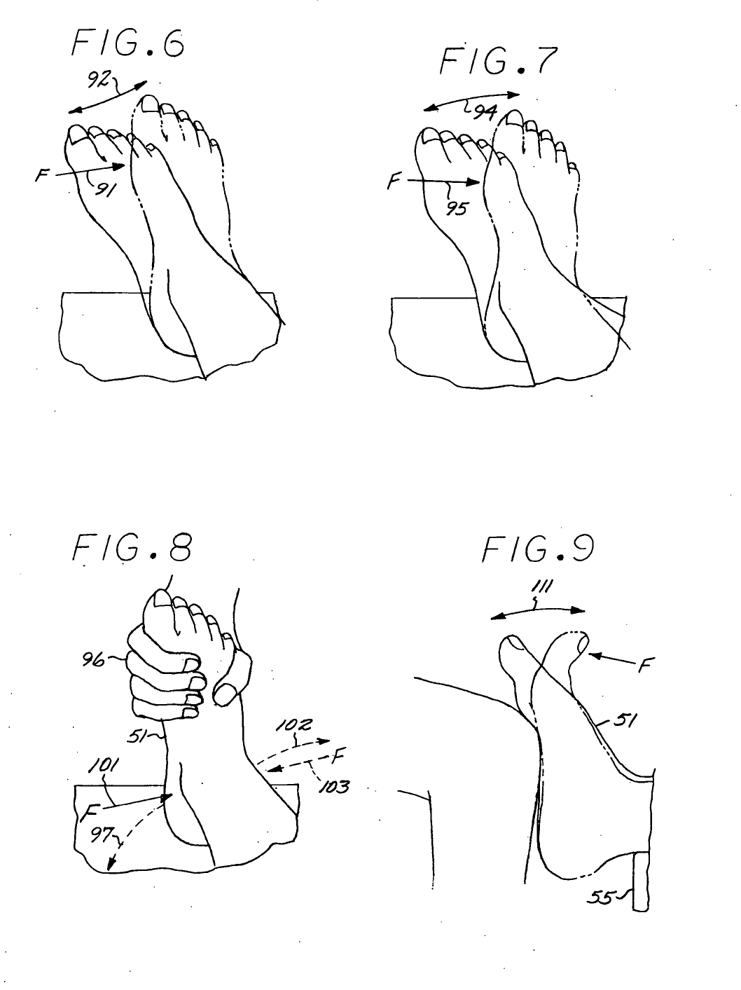

Referring to FIG. 6, in the next steps I might examine flexion for 90° dorsi lateral flexion, invert and then 0° plantar lateral flexion, FIG. 7, while maintaining the foot rested overhanging the stool 55. Here, I direct the patient to rotate the forefoot medially (invert) and then laterally (evert), repeating that movement as shown in FIG. 7 while applying a yieldingly resisting force, a couple of repetitions in the direction of the arrow 92 to warm up that movement as shown in step 57 (FIG. 1). I then press against the medial side of the forefoot in the direction of the force arrow 91 to resist medial movement and maintain that force as the forefoot is rotated back and forth. I observe any segmentation in the movement, to again determine if, and to the extent, there has been decalibration, step 59 (FIG. 1). If I detect decalibration, I record the magnitude thereof in the patient's record and proceed with the calibration step. Again, I instruct the patient to continue slowly moving the forefoot from medial and lateral in a windshield wiper fashion while applying the resistive force which may be in the order of 1 to 2 lbs. and as selected in the manner discussed herein above, step 61 (FIG. 1).

I continue to monitor the movement of the foot as it is moved repeatedly back and forth through the prescribed arc with the resistive force yieldingly applied thereto. After 5 to 10 repetitions I will pay close attention to the foot movement to observe whether or not the segmentation to the motion has been decreased or possibly even eliminated. Upon detecting improvement or that, the segmentation has been totally smoothed out, I will discontinue the calibration step and will observe and record the results in the calibration chart in the patient's records.

I then move onto the 90° dorsi flexion in Step F to evaluate that movement of the foot as shown in FIG. 7 directing the patient to hold the forefoot in the dorsal direction and to sweep back and forth, to invert and evert through the path defined by the directional arrow 94 against the medially acting force depicted by the arrow 95. This movement is repeated to calibrate and allow me to evaluate the existence or extent of decalibration to test and record the results as indicated at steps 61 and 65 of FIG. 1.

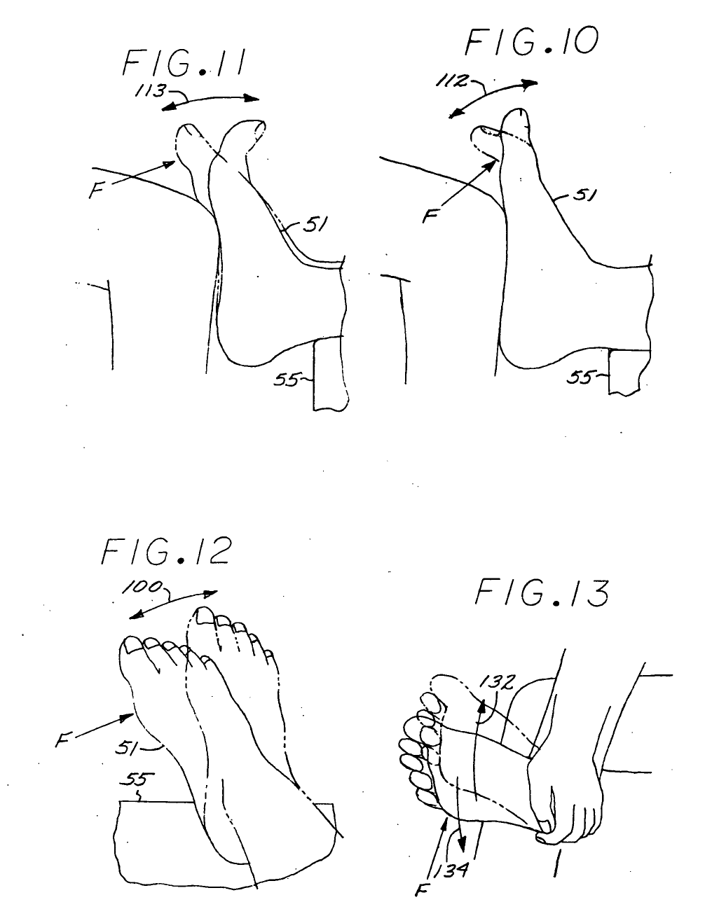

Referring to FIG. 12, I may then direct the patient to maintain the foot supported on the support 55 with plantar flexion and to rotate the forefoot invert and evert in windshield-wiper fashion as indicated by the directional arrow 100 with the low level force applied yieldingly in the lateral direction as indicated by the arrow F.

Referring to FIG. 8, for the next step I will seek to isolate the forefoot by grasping and holding the forefoot with one hand 96 while directing the patient to rotate the heel repeatedly in the medial and lateral direction as indicated by the directional arrow 97, using my other hand to yieldingly resist travel of the heel medially and directing the patient to also resist that fore as he or she rotates the heel laterally by a low level force as indicated by the force arrow 101. After a couple of warm up repetitions in this direction, I will observe the degree and extent of decalibration. If segmented motion is detected indicating decalibration, I then proceed with calibration by continuing to hold the forefoot secure and directing the patient to repeat slow rotations of the heel in the medial and lateral directions (arrow 97); all the while yieldingly applying the resistive force in the medial direction both during the concentric movement laterally and eccentric movement medially for a number of repetitions, for instance from 10 to 15 and up to 20 until I sense there has been a diminishment in the segmentation. At the conclusion of the calibration step, I will record the magnitude of segmented movement in the appropriate line in the Calibration Chart to thus maintain a record which can be compared with the initial evaluation to indicate the degree of success.

Next I will repeat essentially the same procedure, also with the forefoot fixed by the hand 96 and asking the patient to rotate the heel repeatedly medially and laterally while applying a low level yielding force indicated by the broken line arrow 103 to initially detect any segmented movement to be recorded and, if detected, continue with the repetitions of the rotation of the heel in the medial direction per the directional arrow 102 for a number of repetitions. At the conclusion, I will again observe the extent and magnitude of segmented movement and record the indicated degree of calibration.

Referring to FIG. 9, my next step may then involve dorsal toe extension where I position the patient's foot overhanging the edge of the support and pressing the ball of the foot against my knee or other support surface. I then direct the patient to move his or her toes from full plantar flexion to full dorsi flexion back and forth in the direction of the directional arrow 111 with a yieldingly resistive force applied to the dorsal area of the toes as indicated by the directional arrow F. I first direct the patient to conduct some warm up repetitions in this procedure and then, applying the resistive force, I seek to detect any segmentation indicative of decalibration. The results will be recorded in the calibration chart and I then proceed with the calibration. As above, calibration involves repetitions of the toes being moved in the dorsal direction and then the plantar direction and back as against the resistive force approximately 10% of the maximum which the patient might overcome. After 5, 10, 15 or maybe 20 repetitions, the degree of calibration is sensed and recorded in the foot calibration chart.

I may then proceed to toe curls involving plantar flexion with the patient's foot pressed against by knee (FIG. 11) and reciprocating to dorsi flexion as shown in FIG. 10. In this step, I position the patient's foot overhanging the edge of the support 55 pressing the ball of the foot against my knee or other support surface and directing the patient to repeatedly flex the toes back and forth between a dorsal position, as indicated by the directional arrow 112, flexing through a concentric motion to the plantar position shown in broken lines and then back through an eccentric motion to the dorsal position, repeated several times to allow me to apply the usual resistive force F and detect any segmentation suggesting decalibration. Calibration will involve the continued toe curls and uncurling through the path 112 as resisted by the yielding force of my fingers in both directions. Again, the repetitions will be repeated for 5, 10, 15 or so relatively slow cycles, after which I detect the segmentation and record the index of calibration. The index of decalibration is recorded and I move on to the next calibration step.

Referring to FIG. 13, the next step may involve ankle turns invert where I sit the patient on a chair or the like instructing him or her to cross his or her leg with the ankle on the knee of the leg to be treated. I may assist in supporting the shin of the elevated leg on my knee to stabilize and subject the ankle to invert turns as referred to in the foot calibration chart as step U in the Chart of Steps shown in paragraph 0056. I then instruct the patient to rotate his or her forefoot medially and laterally in the direction of arrows 132 and 134 against a medially acting resistive force as depicted by the force arrow F applied to the forefoot, in some instances, stabilizing the heel with my other hand. This technique is repeated a couple of times to allow me to feel any segmentation and record the decalibration index. I then direct the patient to repeat the movement rotating the foot in a concentric motion laterally and in eccentric motion medially while I continue to apply a 10% magnitude resistive force for the usual 5, 10, 15 to 20 repetitions and then record the results.

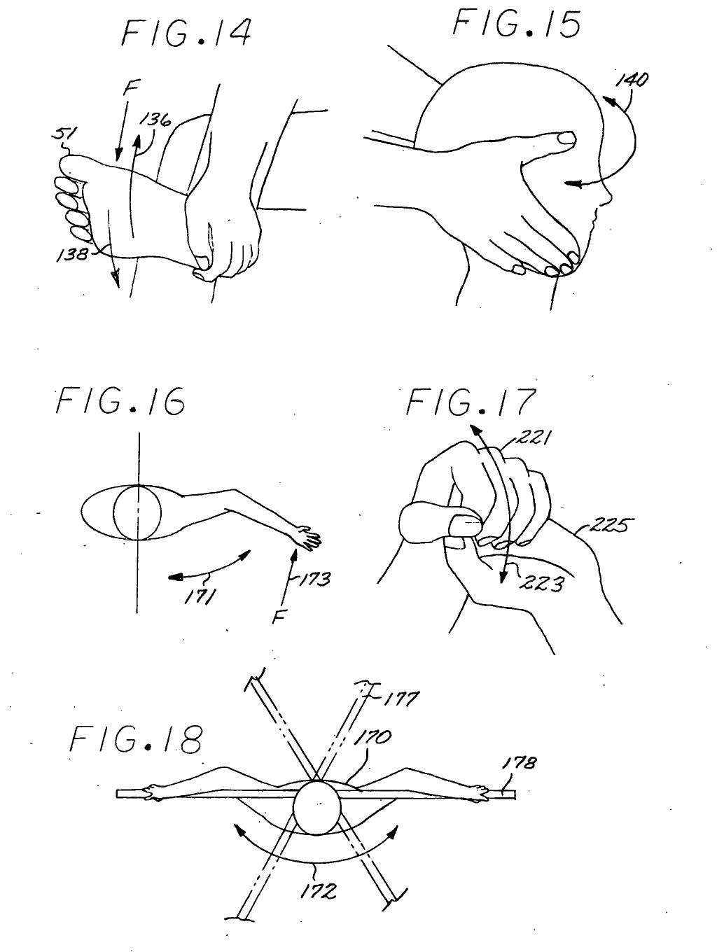

Referring to FIG. 14, my next step may be to execute an ankle turn evert technique where I direct the patient to remain seated and, again with legs crossed and ankle of the subject's foot supported on the other knee of the patient, grasp the heel area in one hand and direct the patient to rotate the ankle, to move the forefoot medially in a concentric fashion and then laterally in an eccentric fashion in the direction of arrows 136 and 138 and repeating that motion against a laterally acting resistive force F applied by my other hand. After a couple of repetitions, I record any segmentation detected and, if indicated, proceed with a number of repetitions for calibration. Again, recording the index indicative of the results.

Referring to FIG. 15, I have discovered that my method is applicable to treatment of the patient's neck. I first apply a passive warm up where by gripping the opposite sides of the patient's head with my palms generally behind the patient's ears and finger tips over the mandible and manipulating the patient's head medially and laterally. I then proceed to a step where the patient rotates his or her head fully to the right and then fully to the left through approximately 180° as depicted by arrow 140. This rotation is resisted by palmer application to the opposite sides of the head resisting rotation in both the clockwise and counterclockwise directions with a force approximately equivalent to 10% of the total force which could be overcome by the patient's neck muscles. This procedure is repeated a couple of times such that I might detect any segmentation and then record in the index. If indicated, I then proceed with the calibration wherein the patient slowly rotates his or her head clockwise and then counterclockwise and at which time I resist those motions by a yieldingly resistive force, again usually the heel of my hand applied generally above the ear and the fingers overlying the mandible in the direction which the patient is turning his or her head and the other hand typically overlying the head area above the ear on the opposite side.

From this disclosure, it will be apparent that a calibration method of the present invention typically involves the patient pushing or turning in one direction and then the other through a selected path as the technician applied a gentle low level resistive force perpendicular to the radius from the axis of rotation. When the movement of the body part results in shortening or contraction of the patient's muscles, this is typically referred to as concentric and when the movement results in lengthening of the muscles, the movement is typically deemed eccentric. To a great degree, the effectiveness of my method focuses on the eccentric phase.

As will be appreciated, my evaluation involves examining the body part(s) to best detect the area and extent of decalibration by examining the body parts for flaws. As an example, my examination may include a relatively light or heavy compression of the plantar tissue along the arches, lateral, medial and transverse, heel and base of the toes as shown as 56 in FIG. 2. My examination may include other examinations of the involved body parts, including the observation of natural motion, detection of particularly sensitive areas and restricted movement or paralysis.

As will be appreciated by those working in the art and referring to FIG. 16, the procedure of the present invention may be applied to joints other than the foot joints such as in the patient's arm, shoulder, elbow, wrist, hand and fingers. In the case of the shoulder, the patient may be instructed to raise his or her arm several times to warm up the muscles and tendons, step 59 (FIG. 1) and then move the arm in the horizontal abduction and adduction directions following directional arrow 171 as I resist that rotation by a force as depicted by the force vector arrow 173 acting rearwardly as the arm is rotated in both directions from the shoulder to benefit from both the eccentric and concentric action of the muscles during movement of the arm. Again, the initial and final movements may be monitored to detect indicators of the degree of decalibration and/or calibration. A similar procedure may be applied for the elbow or wrist.

Referring to FIG. 17, I have also successfully performed the calibration technique of the present invention on the wrist, elbow, fingers and hands 221 of a patient. I perform the technique by both taking a seated position, asking the patient to extend his or her hand 225 fully pronated, palm down, and rotate the hand toward supination (concentric) as indicated by the directional arrow 223, against light resistance indicated by the force applied by my hand 221. Once the patient reaches the end range of motion, if there has been no discomfort, I instruct him or her to reverse the motion to supinate the hand, as against the low level yielding force. This procedure, again, takes advantage of both eccentric and concentric motion against and with a resistive yielding force. I record any segmented movement and then while continuing to apply the low level force instruct the patient to continue repeating the concentric supination an concentric pronation for the usual 5-10 or up to 20 repetitions, periodically seeking to detect any diminishment in the segmented movement, and then recording the index indicative of the results achieved.

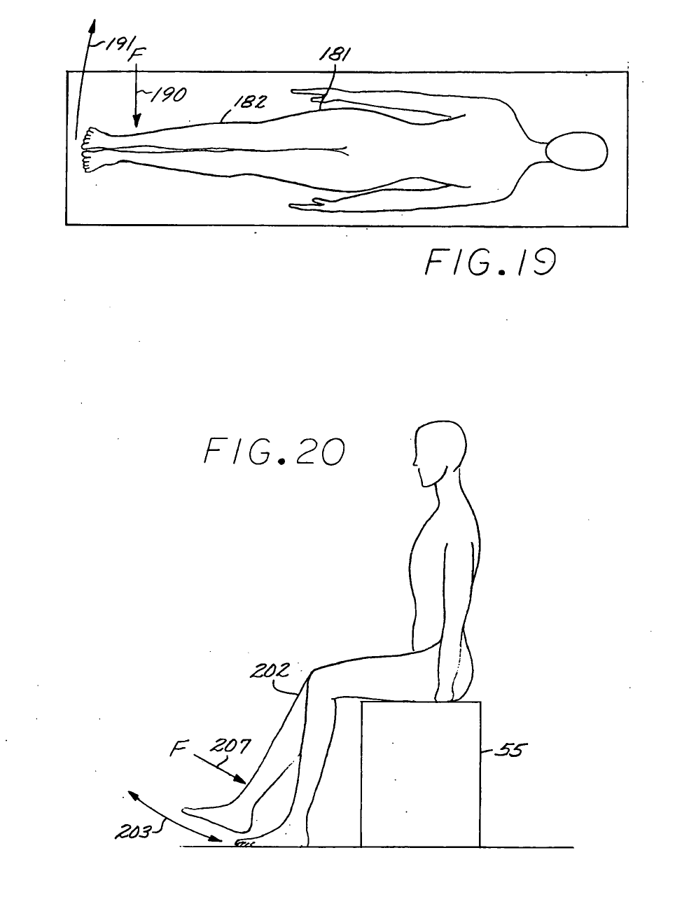

Referring to FIG. 18, I have also found the procedure of the present invention effective for treating decalibration of the waist and torso area. For calibration of the neuromuscular systems controlling these body areas, I address the patient in a standing or seated position and direct him or her to slowly rotate the shoulders 170 from a forwardly facing position all the way to the right and then to the left as indicated by the arrow 172 to warm up the muscles and detect any possible injury or ailment which would indicate that I should not proceed with the procedure. After the warm up steps 57 (FIG. 1), I then direct the patient to rotate his or her shoulders slowly to the right while holding the lower body straight and I may resist the motion by grasping the patient's shoulders to apply a resistive force of maybe 15 lbs. as the motion is repeated. I have discovered a convenient technique where I direct the patient to drape his or her arms over a five or six foot long straight pole 178 rested posterior to the neck and anterior to the elbow as depicted in FIG. 18 and apply the resistance to rotation of that pole. In any event, if decalibration is detected step 59 (FIG. 1), the rotation will be repeated for several repetitions. Again, the initial results may be recorded and I may then direct the patient to rotate the shoulders fully from right to the left and back as indicated by the directional arrow 172 for warm up and then to be rotated back and forth through that arc against a resistive counter clockwise force applied by hand to the pole 178 throughout both directions of movement to calibrate the neuromuscular system and the results recorded.

Referring to FIG. 19, another significant area which may be treated by the method of the present invention is the hip area 181 of the patient. In treating this condition, I request the patient to lay down face up or face down and to move his or her leg 182 forwardly, rearwardly or laterally in a repeated pattern as indicated by the directional arrow 191. I then apply a medially acting low level resistive force as illustrated by the arrow 190 as the patient repeats the motion through numerous repetitions up to 20 until calibration step 61 (FIG. 1) is indicated.

Referring to FIG. 20, I have also found this procedure of the present invention effective in treating the patient's knee, 202. In this treatment, the patient may sit on the support 55 and will be instructed to lift the lower leg up 202 and down in the direction of the directional arrow 203 to warm the knee up. I then apply a resistive force to movement in both directions as indicated by the vector arrow 207. If stutter movement suggests decalibration is in order, I then continue the procedure for up to twenty repetitions or as dictated for proper calibration step 65 (FIG. 1).

From the foregoing, it will be apparent that the Neuromuscular Calibration procedure of the present invention provides a major benefit in the treatment of segmented movement in patient's body parts. In my experience, the procedure often serves to enhance patient skills through the increase of finer proprioceptive capacity. A hand calibration appears to allow the patient's to, after the treatment, receive more information through their hands to thus enhance their physical skills. The results oftentimes appear to be relatively instantaneous with the application of the procedure. The benefits to the musculoskeletal system are oftentimes include a reduction or elimination of pain, relatively rapid recovery from injury, restored or improved joint function and range of motion, optimization of muscle engagement and frequently increases the ability of the patient to resist injury. There seems to be minimal recidivism since the calibrated body parts are relatively resistant to reversal. Progress often continues after the treatment and can be seen to improve flexibility, balance, coordination and strength. In some instances, the procedure will restore functionality to body parts which have previously been experienced as paralyzed.

1. A method of treating a patient's articulated body part comprising:
evaluating the patient by an oral interview and physical observation to identify symptoms;
recording the symptoms in a record;
subjecting the articulated body part to manipulation for a passive warm up;
mobilizing the articulated body part by passive stretching and manual compression of the body part tissue;
assessing the body part condition by directing the patient to move the articulated body part slowly back and forth through a selected prescribed path while yieldingly applying a low level resistive force to be resisted by the patient's neuromuscular system as the part moves in at least one direction through the path;
sensing movement of the body part as it moves through to detect any segmented motion;
recording in the record data indicative of the presence or absence of any segmented motion detected;
conducting a plurality of calibration steps by; A) directing the patient to repeatedly move the articulated body part concentrically and eccentrically through the prescribed path; B) while yieldingly applying a resistive force acting in one direction along the prescribed path at a magnitude sufficient to engage the neuromuscular system; and
thereafter, directing the patient to move the body part through the prescribed path while observing any segmented motion and recording the results in the record.

2. The method claim 1 for treating the patient's foot and wherein:
the passive warm up includes passive motion of selected parts of the patient's foot;
the mobilization includes passive stretching of selected foot parts;
the calibration includes moving a foot part under low level yielding resistance through the selected path of motion.

3. The method set forth in claim 1 for treating the patient's foot and that wherein:
the evaluation includes instructing the patient to walk a selected number of steps and observing the walking motion and recording in the record any asymmetrical motion observed.

4. The method of claim 1 for treating the patient's foot and wherein:
the calibration step includes, placing the back of the patient's heel on a support, instructing the patient to curl his or her toes back and forth plantar and dorsi through the prescribed path while yieldingly resisting movement of the toes in at least the one direction.

5. The method of claim 4 for treating a patient's foot and wherein:
in the calibration steps, the force is applied to the dorsi surface of the toes.

6. The method of claim 1 for treating the patient's foot wherein:
the calibration includes, while resting the patient's leg on a support, instructing the patient to repeatedly rotate the forefoot plantar and doris through the prescribed path while applying the resistive force against the plantar surface of the forefoot.

7. The method of claim 1 for treating the patient's foot and wherein:
the calibration includes, while resting a patient's heel on a support, instructing the patient to repeatedly rotate the forefoot back and forth dorsi and plantar through the prescribed path while applying the resistive force to the dorsi surface of the forefoot.

8. The method of claim 1 for treating the patient's foot and wherein:
the calibration includes, while resting the patient's heel on a support, fixing the forefoot against movement and selectively rotating the heel repeatedly back and forth invert and evert through the prescribed path while applying the resistive force opposing the rotation in at least one direction.

9. The method of claim 1 for treating the patient's foot wherein:
the calibration includes instructing the patient to, while sitting, cross his or her ankle over the other knee and, while holding the heel, instructing the patient to rotate the forefoot invert and evert through the path, while applying the force to one side of the foot.

10. The method of claim 1 for treating the patient's foot and wherein:
the calibration includes instructing the patient to repeatedly curl the toes plantar and dorsi while applying a yieldingly resistive force to the dorsi surface of the toes for a plurality of repetitions and then repeating curling step while applying a yieldingly resistive force to the plantar surface of the toes.

11. The method of claim 1 for treating a patient's foot and wherein:
the calibration includes fixing one end of the foot and instructing the patient to rotate the other end of the foot repeatedly invert and evert while applying the resistive force against the other end of the foot, first medially for a plurality of repetitions and then laterally for a plurality of repetitions.

12. The method of claim 1 for treating the patient's foot and wherein:
the calibration includes, while applying the resistive force to resist plantar movement of the lateral portion of the foot, instructing the patient to seek to repeatedly move the lateral portion of the foot plantar against the resistive force.

13. The method of claim 1 for treating the patient's foot and wherein:
the calibration step includes applying the resistive force against the medial portion of the plantar surface of the patient's forefoot and instructing the patient to repeatedly move the medial portion of the forefoot plantar against the resistive force.

14. The method of claim 1 for treating the patient's neck and wherein:
the calibration step includes instructing the patient to rotate his or her head back and forth at a constant speed while applying the restrictive force thereto to yieldingly resist rotation in at least one direction.

15. The method of claim 1 for treating the patient's neck and wherein:
the calibration step includes instructing the patient to repeatedly rotate his or her head back and forth while applying the restrictive force to resist rotation in both directions.

16. The method of claim 1 for treating the patient's shoulder and wherein:
the calibration step includes instructing the patient to repeatedly move his or her arm back and forth away from the trunk through the prescribed path while applying the restrictive force to resist movement in at least one direction.

17. The method of claim 1 for treating the patient's arm that includes:
instructing the patient to move a portion of the arm back and forth through the prescribed path about an articulation joint while applying the restrictive force to restrict the patient's neuromuscular system controlling movement of the portion of the arm through the prescribed path.

18. The method of claim 1 for treating the patient's torso and wherein:
the calibration step includes directing the patient to repeatedly rotate the upper torso back and forth through the prescribed path while applying the resistive force to rotation in at least one direction.

19. The method of claim 1 that includes:
placing a pole over the patient's shoulders, behind the neck, and instructing the patient to rotate his or shoulders back and forth, right and left, while applying the force to the pole.

20. The method of claim 1 for treating the patient's hip wherein:
the calibration step includes instructing the patient to repeatedly move his or her leg back and forth laterally or medially while applying the restrictive force to a distal extremity.

21. A method of treating a patient's foot comprising:
while the patient is walking, observing the patient and recording in a record data in a record indicative of any asymmetrical walking movements;
taking the patient's medical history and recording the history in the record;
observing the data recorded in the record;
after the observation, applying a passive warm up to the patient by causing the patient to move selected foot parts relative to other foot parts;
causing the selected foot parts to be moved, stretched and compressed; and
calibrating the neuromuscular system involved with the selected foot parts by causing the patient to activate the neuromuscular system to move the selected foot parts repeatedly back and forth through respective prescribed motion paths while yieldingly applying respective low level resistive forces in at least one direction against the selected foot parts as they are moved through the respective paths.

22. A method of treating a patient's foot comprising:
supporting the foot on a support with the forefoot extending upwardly;
calibrating the neuromuscular system for the foot by applying a yielding force to the plantar surface of the forefoot and instructing the patient to repeatedly rotate the forefoot inward (invert) and outward (evert) through a prescribed path;
fixing the forefoot and rotating the heel back and forth medially and laterally relative to the forefoot while yieldingly applying a low level force to one side and then the other of the heel;
fixing the heel and causing the patient to rotate the forefoot laterally and medially relative to the heel while yieldingly applying low level force to movement of the forefoot first in the lateral direction for a selected number of rotations and then from the medial direction for a plurality of rotations;
causing the patient to curl his or her toes downwardly and, while curled, causing the patient to rotate the forefoot laterally while applying a low level force to lateral movement and then directing the patient to rotate the forefoot repeatedly medially while applying a laterally acting force to the forefoot;
causing the patient to fold his or her toes distally and while maintaining them folded, rotating the forefoot repeatedly back and forth medially laterally a plurality of times, first resisting rotation by yieldingly applying a resistive force medially and then resisting rotation by yieldingly applying a resistive force laterally; and
directing the patient to curl his or her toes medially and while curled, to rotate the forefoot back and forth medially and laterally through the prescribed path while yieldingly applying a restrictive force to resist lateral rotation for a selected number of rotations and then applying a restrictive force to resist rotation medially for a plurality of rotations.
